(19) United States
(12) Patent Application Publication
(10) Pub. No.: US 2024/0358618 A1
(43) Pub. Date: Oct. 31, 2024

(54) NAIL POLISH TOPCOAT COMPOSITION

(71) Applicant: L'OREAL, PARIS (FR)

(72) Inventors: Luke Zachary SHAPIRO, Matawan, NJ (US); Ramakrishnan HARIHARAN, Springfield, NJ (US); I-fan HSIEH, Scotch Plains, NJ (US)

(73) Assignee: L'OREAL, PARIS (FR)

(21) Appl. No.: 18/309,156

(22) Filed: Apr. 28, 2023

(51) Int. Cl. A61K 8/73; A61K 8/81; A61Q 3/02

(52) U.S. Cl. CPC A61K 8/731; A61K 8/8147; A61Q 3/02; A61K 2800/30

(57) ABSTRACT

A composition for use on the nails is provided. The includes a solvent system that includes at least one volatile, non-aqueous polar solvent; a water-insoluble nitrocellulose polymer; an acrylic copolymer with an acid value ranging from about 145 and about 170 mg KOH per gram; and a cellulose ester of at least one C2-C4 organic acid. Methods including those for use as a topcoat are also provided.

### FIELD OF THE INVENTION

The present invention relates to nail polish topcoat compositions, and specifically to nail polish topcoat compositions comprising certain acrylic polymers.

### BACKGROUND

Consumers use nail polish to cosmetically enhance the appearance of their nails or protect the nails from the abuses found in their everyday environment. Often this requires a nail polish that is formulated to provide a good shine.

Consumers also desire a durable nail polish. Lack of durability is often evidenced by unsightly chipping or peeling of the coating soon after the original coating has been applied, requiring at least in part a reapplication of the coating in an attempt to recreate the aesthetic appearance of the original nail coating. Durability is often a delicate balance, as coatings that are too soft can scuff and coatings that are too stiff suffer from chipping.

A continuing need exists to simultaneously provide both improved gloss and improved durability, particularly for nail polish topcoats.

### BRIEF SUMMARY

The present invention is directed to a nail polish topcoat composition (also referred to herein as “nail topcoat composition,” or just “composition”). The nail polish topcoat composition includes a solvent system. The solvent system includes at least one volatile, non-aqueous polar solvent. The nail polish topcoat composition further includes a water-insoluble nitrocellulose polymer as well as an acrylic copolymer with an acid value ranging from about 145 and about 170 mg KOH per gram. The nail polish topcoat composition further includes a cellulose ester of at least one C2-C4 organic acid.

Advantageously, the acid number of the acrylic polymer may be between about 160 and about 170 mg KOH per gram, and/or be present in a concentration by weight in the nail topcoat composition that is from about 0.5% to about 15% by weight.

It may also be advantageous for the cellulose ester of at least one C2-C4 organic acid to be or include cellulose acetate butyrate and/or be present in a concentration by weight in the nail topcoat composition that is from about 1% to about 3%.

It may also be advantageous for the composition to be substantially free of colorant.

It may also be advantageous for the at least one volatile, non-aqueous polar solvent to include at least one volatile acetate, such as ethyl acetate and butyl acetate, such as where these compounds are present in a concentration by weight such that a total concentration by weight of ethyl acetate and butyl acetate in the nail topcoat composition is from about 50% to about 90%.

It may also be advantageous for the nail topcoat composition to further include sucrose acetate butyrate and/or acetyl tributyl citrate.

It may also be advantageous for the water-insoluble nitrocellulose polymer to be nitrocellulose such as to be present in a concentration by weight in the nail topcoat composition that is from about 5% to about 15%.

According to certain embodiments, the nail polish topcoat composition may meet a plurality or even all of these above listed requirements.

According to another aspect of the invention, a method of treating a nail is provided. The method includes applying to the nail the above-described nail polish topcoat composition.

### BRIEF DESCRIPTION OF THE DRAWINGS

Implementation of the present technology is described, by way of example only, with reference to the attached FIGURE, wherein:

FIG. 1 shows a graph of hardness over time for various compositions.

### DETAILED DESCRIPTION

As used herein, the expression “at least one” means one or more and thus includes individual components as well as mixtures/combinations.

Other than in the operating examples, or where otherwise indicated, all numbers expressing quantities (e.g., concentrations, ratios, and the like) of ingredients and/or reaction conditions are to be understood as being modified in certain embodiments by the term “about,” meaning within 10% to 15% of the indicated number (e.g. “about 10%” means 8.5% to 11.5% such as 9% to 11%, and “about 2%” means from 1.7% to 2.3 such as from 1.8% to 2.2%).

Similarly, for ratios, the modifier “about” means within 10% or 15% of the number. For example, about 4:1 means from 3.4:1 to 4.6:1, preferably 3.6:1 to 4.4:1. As readily understood by one skilled in the art, where the first ingredient in a ratio is less than the second, then a ratio may be expressed “inversely.” For example, if a second ingredient, B is present in an amount or concentration that is 2.5 times greater than that of ingredient A, this may be identified as an A:B ratio of 1:2.5. “About 1:10,” means from 1:8.5 to 1:11.5, preferably 1:9 to 1:11. As one skilled in the art will understand, the modifier “about” also encompasses the exact number or ratio specified. Unless otherwise indicated, all concentrations shown as percentages are concentrations by weight and also, unless otherwise indicated, relate to the entire nail topcoat composition as a whole.

As used herein, articles such as “a” and “an” when used in a claim, are understood to mean one or more of what is claimed or described.

As used herein, the term “at least one” means one or more and thus includes individual components as well as mixtures/combinations.

As used herein, the terms “include”, “includes” and “including” are meant to be non-limiting.

“Film-former” or “film forming agent” or “film forming polymer” or “film forming resin” as used herein mean a polymer or resin that leaves a film on the substrate to which it is applied, for example, after a solvent accompanying the film-former has evaporated, absorbed into and/or dissipated on the substrate. A “silicone film-former” is a film-former that includes at least one silicone (a silicone atom bonded directly to an oxygen atom and also to a carbon atom such as in an organic moiety).

“Liquid” or “liquid cosmetic” or “liquid lipstick” or “liquid composition” means a composition having a fixed volume, flows to cover the bottom and assumes the shape of the portion of the container it fills and is slightly compressible (as disclosed in General chemistry, Fourth Edition 2005, p. 434. This is meant to exclude conventional stick-based lipsticks that are formed by molding and do not flow as a liquid would.

“Substituted” as used herein, means comprising at least one substituent. Non-limiting examples of substituents include atoms, such as oxygen atoms and nitrogen atoms, as well as functional groups, such as amine groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Substantially free” as it is used herein means that while it is preferred that no amount of the specific component be present in the composition, it is possible to have small amounts of it in the compositions of the invention provided that these amounts do not materially affect at least one, preferably most, of the advantageous properties of the compositions of the invention. In certain embodiments, substantially free means less than about 2% of the identified ingredient, such as less than about 1%, such as less than about 0.5% based on the composition as a whole (or a particular component, if indicated). The term “anhydrous” means substantially free of water.

Numerical ranges are inclusive of endpoints and meant to include all combinations and sub-combinations. For example, from about 5%, 10% or 15% to about 20%, 50% or 60% may refer to about 5% to about 20%, about 5% to about 50%, about 5% to about 60%, about 10% to about 20%, about 10% to about 50%, about 10% to about 60%, about 15% to about 20%, about 15% to about 50%, or about 15% to about 60%. As used herein a range of ratios is meant to include every specific ratio within, and combination of subranges between the given ranges.

The compositions and methods of the present invention can comprise, consist of, or consist essentially of the essential elements and limitations of the invention described herein, as well as any additional or optional ingredients, components, or limitations described herein or otherwise useful.

Nail topcoat compositions of the present invention include a solvent system, a water-insoluble nitrocellulose polymer; an acrylic polymer; and a cellulose ester of at least one C2-C4 organic acid. Each of these will be discussed in turn.

### Solvent System

Nail topcoat compositions of the present invention include a solvent system. The function of the solvent system is to dissolve or disperse various components in the nail topcoat composition and to evaporate at an appropriate rate in order to allow for the relatively fast formation of film that has desirable gloss and durability properties.

The solvent system includes at least one volatile, non-aqueous polar solvent. The volatile, non-aqueous polar solvent is (1) “volatile” in that it evaporates at a rate at least as fast as butyl acetate; (2) is polar in that it includes at least one polar functional group (e.g., alcohol, ether, ester, ketone, hydroxyl, amine, amino, carboxyl, carbonyl, and the like), and (3) “non-aqueous” in that it is not water, and, in certain embodiments is not completely miscible in water throughout all concentrations at standard temperature and pressure.

According to certain embodiments, the at least one volatile, non-aqueous polar solvent includes an ester, such as one or more acetate compounds. In particularly notable embodiments, the acetate compound is selected from ethyl acetate, butyl acetate, and combinations thereof. In certain embodiments the one or more acetate compounds are used as a majority portion of the at least one volatile, non-aqueous polar solvent, meaning that the proportion by weight of the one or more acetate compounds in the total amount at least one volatile, non-aqueous polar solvent is more than 50%, such as more than about 75%, such as more than about 90%.

According to certain embodiments, the at least one volatile, non-aqueous polar solvent is present in the nail topcoat composition in a concentration by weight that is from about 50% or 60% to about 75%, 80% or 90%. In notable embodiments, the at least one volatile, non-aqueous polar solvent comprises, has a majority fraction (and more notably at least about 90% of the total volatile, non-aqueous polar solvents) that is a mixture of volatile acetates such as ethyl acetate and butyl acetate. In other notable embodiments, the nail topcoat composition has a total concentration by weight of ethyl acetate and butyl acetate in the nail topcoat composition is from about 50% to about 90%.

According to certain other embodiments the at least one volatile, non-aqueous polar solvent includes one or more monoalcohols, such as C2-C5 monoalcohol such as ethanol or isopropanol. In certain embodiments the one or more monoalcohols are used as a minority portion of the at least one volatile, non-aqueous polar solvent, meaning that the proportion by weight of the one or more monoalcohols in the total amount at least one volatile, non-aqueous polar solvent is less than 50%, such as less than about 25%, such as less than about 10%.

### Water-Insoluble Nitrocellulose Polymer

Nail topcoat compositions of the present invention include a water-insoluble nitrocellulose polymer (e.g., cellulose nitrate). The nitrocellulose polymer may be formed by nitrating cellulose by exposure to acids such as nitric acid and/or sulfuric acid. Desirably the nitrocellulose is soluble in the solvent system and more particularly soluble in the one or more volatile, non-aqueous polar solvents. The water-insoluble nitrocellulose polymer serves as a film-former in the nail topcoat composition. The nitrocellulose may be present in a concentration by weight in the nail topcoat composition that is from about 5%, 6% or 7% to about 10%, 12% or 15% by weight.

Acrylic Polymer Having Acid Number from about 145 Mg KOH Per Gram to about 170 Mg KOH Per Gram

Nail topcoat compositions of the present invention include an acrylic (co) polymer having an acid number (also known as acid value) that ranges from about 145 mg KOH per gram and about 170 mg KOH per gram. Acid value or acid number is reflective of the relative amount of carboxyl groups in the acrylic (co) polymer and is generally measured using titration, as known to one skilled in the art. The acrylic (co) polymer having an acid number will be formed from one or more types of ethylenically unsaturated monomeric substituents.

According to certain embodiments, the acid number of the acrylic polymer may be from about 160 to about 170 mg KOH per gram.

According to certain other embodiments the acid number of the acrylic polymer may be greater than 150 mg KOH per gram, such as ranging from greater than 150 to about 170 mg KOH per gram.

According to certain other embodiments, the acid number of the acrylic polymer is from about 145, 146, 147, 148, 149, 150 or 160 mg KOH per gram to about 160 or 170 mg KOH per gram.

The present inventors have found that use of these relatively high acid value acrylates provide a combination of beneficial properties, particularly when used in combination with other formulation components detailed herein.

The acrylic (co) polymer having an acid number that ranges from about 145 and about 170 mg KOH per gram may have an INCI designation of, for example, Acrylates Copolymer.

In certain embodiments, the acrylic polymer having an acid number that ranges from about 145 and about 170 mg KOH per gram may have a softening point, as measured by the Ball & Ring method, known to those skilled in the art, that ranges from 110° C. to about 125° C.

Suitable acrylic polymers having acid number that ranges from about 145 mg KOH per gram and about 170 mg KOH per gram include those available from Estron Chemical, Inc of Calvert City, KY and sold under the trade names EPOMATT G-152, EPOMATT G-154 or ISOCRYL G-265.

The acrylic copolymer with an acid value ranging from about 145 and about 170 mg KOH per gram is present in a concentration by weight in the nail topcoat composition that is from about 0.5%, 1% or 2% to about 5%, 10% or 15% by weight.

### Cellulose Ester of at Least One C2-C4 Organic Acid

Nail topcoat compositions of the present invention include a cellulose ester of at least one C2-C4 organic acid. The cellulose ester of at least one C2-C4 organic acid may be useful to plasticize the resulting film formed from the nail top topcoat composition. Examples of suitable cellulose esters include those soluble in C2-C4 alcohols and/or ethyl acetate. According to certain embodiments, the C2-C4 organic acid is or includes acetic acid and butyric acid. According to certain embodiments the cellulose ester of at least one C2-C4 organic acid is or includes cellulose acetate butyrate (“CAB”).

The cellulose ester of at least one C2-C4 organic acid may be, for example, cellulose acetate butyrate commercially available from Eastman Chemical such as CAB-381-0.5.

The cellulose ester of at least one C2-C4 organic acid may be present in a concentration by weight in the nail topcoat composition that is from about 0.5%, or 1% to about 2% 3%, or 5%.

According to certain embodiments, the acrylic copolymer with an acid value ranging from about 145 and about 170 mg KOH per gram and the cellulose ester of at least one C2-C4 organic acid may be present in a ratio of concentration by weight of acrylic copolymer with an acid value ranging from about 145 and about 170 mg KOH per gram to concentration by weight of cellulose ester of at least one C2-C4 organic acid (e.g., CAB) that is in a range from about 0.5:1, 1:1, 1.5:1, 2.0:1 to about 3.0:1, 4.0:1, 5.0:1 or 10:1.

### Other Ingredients

Nail topcoat compositions of the present invention may include various other ingredients without significantly compromising the beneficial properties.

For example, according to certain embodiments, plasticizers other than the cellulose acetate butyrate may be included in the nail topcoat composition. Examples of such other plasticizers include polar molecules having multiple ester groups such as those having a molecular weight in a range from about 250 or 400 to about 1000 or 1500. Examples include sucrose acetate butyrate and acetyl tributyl citrate. In certain embodiments the nail topcoat composition includes an ingredient selected from a group consisting of sucrose acetate butyrate and acetyl tributyl citrate. Acetyl tributyl citrate is sold under Citroflex brand by Vertellus, or the Uniplex brand by Unitex Chemical of Greensboro, North Carolina.

Other exemplary plasticizers include dipropylene glycol dibenzoate, and 1,2-propylene glycol dibenzoate, trioctyl citrate, triethyl citrate, acetyl tributyl citrate, or tributyl citrate. Sucrose acetate butyrate is sold under the name SAIB 90 by Eastman Chemical of Kingsport, TN.

These other plasticizers may be present in concentrations ranging from about 1%, 2% or 3% to about 10%, 15% or 25% by weight.

As another example, according to certain embodiments, additional polymers or resins may be included in the nail topcoat composition. One example of a suitable additional resin or polymer are alkyd resins. An example of a suitable alkyd resin is a phthalic anhydride/glycerin/glycidyl decanoate copolymer, such as ALUKIDIR ODE 230 70 E available from Dainppon Ink & Chemicals of Tokyo, Japan. Another example of a suitable resin is a ketone-aldehyde resin, e.g., a copolymer comprising an aromatic ketone. Potential ketone-aldehyde resins may include, but are not limited to, those described in U.S. Pub. No. 2009/0012245, U.S. Pat. Nos. 7,183,372 and 7,101,958, or those sold by Evonik Industries under the TEGO® Variplus brand, such as TEGO® Variplus SK.

As another example, according to certain embodiments, mattifying agents may also be included in the nail topcoat composition. Examples include silica, aluminosilicates, borosilicates, and the like. These mattifying agents may be present in concentrations ranging from about 0.25%, 0.5% or 1% to about 1%, 2% or 5% by weight.

As another example, according to certain embodiments, hydrocarbon oils may also be included in the nail topcoat composition. “Hydrocarbon oil” refers to a compound which is liquid at room temperature, is insoluble in water and is of hydrophobic character. The hydrocarbon oils used in conformity with the invention are typically synthetic oils, mineral, vegetable or animal oils, unsaturated fatty alcohols, and esters of fatty acids and lower C2-C4 mono- or polyalcohols.

The one or more hydrocarbon oils may be present in an amount ranging from about 1% to about 20% by weight, more preferably from about 1% to about 10% by weight, and still more preferably from about 0.5% to about 5% by weight.

As another example, according to certain embodiments, non-polar silicone copolymers may be included in the nail topcoat composition. In certain embodiments, the non-polar silicone copolymer may comprise repeating units of at least one polar (meth)acrylate unit and vinyl copolymers grafted with at least one non-polar silicone chain. Non-limiting examples of such copolymers are acrylates/dimethicone copolymers such as those commercially available from Shin-Etsu, for example, the product sold under the tradename KP-543 or KP-545, or acrylates/stearyl acrylate/dimethicone acrylates copolymers, such as those commercially available from Shin-Etsu, for example, the product sold under the tradename KP-561, and acrylates/behenyl acrylate/dimethicone acrylates copolymer, such as those commercially available from Shin-Etsu, for example, the product sold under the tradename KP-562.

The non-polar silicone copolymer may present in an amount ranging from about 0.01% to about 2% by weight. Preferably, the non-polar silicone copolymer may present in an amount ranging from about 0.01% to about 1% by weight, more preferably from about 0.05% to about 0.6% by weight.

In certain notable embodiments, the nail polish topcoat composition may include certain components such as water and/or colorants (coloring agent). However in other embodiments, it is generally preferred that the nail topcoat composition be substantially free of water and/or colorants.

The colorants that the nail topcoat composition may be substantially free of include dyes, pigments, pearls, effect pigments and the like that are commonly used in color cosmetics or in color coat nail polishes.

Dyes include, for example, fat-soluble dyes such as Sudan red, DC Red 17, DC Green 6, β-carotene, Sudan brown, DC Yellow 11, DC Violet 2, DC Orange 5 and quinoline yellow.

Pigments can be white or colored, inorganic and/or organic and coated or uncoated. Mention may be made, for example, of inorganic pigments such as titanium dioxide, optionally surface treated, zirconium or cerium oxides and iron or chromium oxides, manganese violet, ultramarine blue, chromium hydrate and ferric blue. Mention may also be made, among organic pigments, of carbon black, pigments of D & C type and lakes based on cochineal carmine or on barium, strontium, calcium or aluminum, such as D&C Red No. 10, 11, 12, and 13, D&C Red No. 7, D&C Red No. 5 and 6, and D&D Red No. 34, as well as lakes such as D&C Yellow Lake No. 5 and D&C Red Lake No. 2.

Pearlescent pigments include, for example, white pearlescent pigments, such as mica covered with titanium oxide or with bismuth oxychloride, colored pearlescent pigments, such as titanium oxide-coated mica with iron oxides, titanium oxide-coated mica with in particular ferric blue or chromium oxide, or titanium oxide-coated mica with an organic pigment of the abovementioned type, and pearlescent pigments based on bismuth oxychloride.

Nail topcoat compositions of the present invention may be made by methods known in the art, such as by optionally premixing certain ingredients into the one more solvents and blending various ingredients together in careful manner to provide a homogeneous mixture.

### Methods of Use

Nail topcoat compositions may be applied onto a nail such as human fingernail or toenail. According to certain embodiments the nail has a previously applied color coat formed thereon and the nail topcoat composition is applied atop the previously applied color coat formed on the nail. The nail topcoat composition may be applied onto the nail/previously applied color coat formed thereon by commonly used methods such as brushing or spraying.

The following examples are intended to illustrate the invention without limiting the scope as a result. The percentages are given on a weight basis.

### EXAMPLES

### Example 1: Flexibility Testing

Compositions were prepared to evaluate the flexibility of films formed from five polymeric materials. Specifically the following materials were evaluated: (1) an acrylic copolymer having an acid value between 130 mg KOH per gram and 150 mg KOH per gram [Example C1]; (2) an acrylic copolymer having an acid value between 145 mg KOH per gram and 165 mg KOH per gram [Example E1, consistent with embodiments of the invention]; (3) an acrylic copolymer having an acid value between 160 mg KOH per gram and about 170 mg KOH per gram [Example E2, consistent with embodiments of the invention]; (4) cellulose acetate butyrate [Example C2]; (5) an acrylic copolymer having an acid value of 0 mg KOH per gram [Example C3].

A test solution of each of the materials above was prepared by dissolving various materials to a concentration by weight in ethyl acetate of 40% (except for Example C3, which was 50% active). The test solutions were evaluated using a conical mandrel test, ASTM test D522-93a (2008), “Standard Test Methods for Mandrel Bend Test of Attached Organic Coatings.”

Cold-rolled steel test strips are used as substrates (22 gauge, 0.8 mm thick), preferably 100 mm×150 mm long/wide. The strips are cleaned such as with volatile solvent, to remove residual oil. Uniform coatings are applied to the test strips and allow them to air dry. The coated dried samples are conditioned for at least about 2 hrs. and 50% relative humidity. A conical mandrel tester having a smooth steel 200 ml long cone with a diameter of 3 mm at one end and 38 mm at the other end is employed.

With the operating lever of the apparatus in a horizontal position, the test specimen is slipped between the mandrel and the drawbar with the finish side towards the drawbar. The specimen is rigidly clamped in a vertical position adjacent to the mandrel by placing the long edge behind the clamping bar in such a manner that the panel is always set up to the narrow end of the mandrel.

The lever is moved through about 180° at uniform velocity to bend the specimen approximately 135°.

To determine crack resistance under more simulated use conditions, the bend time should be about 1 s. The bent surface of the specimen is examined immediately with the unaided eye for cracking.

The results of the test indicated that Example C1 showed some cracking after day 1; Example C2 showed cracking after day 1; Example C3 showed some cracking and additionally showed significant buckling after day 1; Example E1 and Example E2 showed minimal cracking.

### Example 2: Hardness

Compositions were prepared to evaluate the hardness of films formed from three polymeric materials. Specifically the following materials were evaluated: (1) an acrylic copolymer having an acid value between 130 mg KOH per gram and 150 mg KOH per gram [Example C4]; (2) an acrylic copolymer having an acid value between 145 mg KOH per gram and 165 mg KOH per gram [Example E3, consistent with embodiments of the invention]; (3) an acrylic copolymer having an acid value between 160 mg KOH per gram and 170 mg KOH per gram [Example E4, consistent with embodiments of the invention].

A test solution of each of the materials above was prepared dissolving various materials to a concentration by weight in ethyl acetate of 40%. The test solutions were evaluated at various time points after application to a glass plate with 6 mil thickness film (two coats). Hardness was evaluated using a Persoz pendulum hardness reader.

To perform the test, a 5×4 inch glass plate and a 6 mil Bird applicator are thoroughly cleaned with acetone and wiped KLEENEX tissue until dry. The glass plate is placed in the center of an automatic draw down machine of a Erichsen Dry Time Apparatus. The speed of the drawdown machine is set for 25 mm/sec. The Bird Applicator is set on the glass plate and flush against the moving bar. Three ml of test composition is placed across the width of the glass plate. The glass place is held and the start button is pressed on the drawdown machine to form a homogeneous film across the length of the plate without moving the position on the plate. The film is drawn down using the 6 mil Bird Applicator. The drawdown bar is reset and residue is cleaned from glass plate and drawdown machine with acetone. The film is allowed to dry about 24 hours (1 day) and initial hardness reading is taken. Additional measurements are taken at 5, 7, 10, and 14 days after initial draw down.

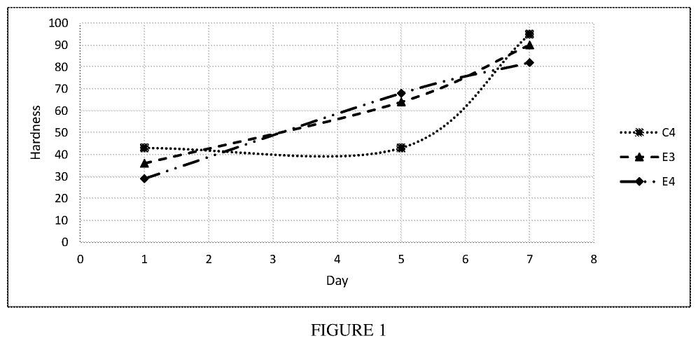

As shown in FIG. 1, the results of the test indicated that Example E3 and Example E4 showed a fairly linear hardness over time. In contrast, Example C4 showed a non-linear hardness over time—with days 1-5 showing relatively low increase in hardness, followed by a sharp rise and rising above the hardness of Examples E3 and E4. The hardness profile of Example C4 is in some instances less than desirable, since it stays soft (and therefore subject to deformation) particularly through day five and then becomes quite high (then potentially subject to chipping).

### Example 3: Gloss

Nail topcoat compositions were prepared to evaluate the shine or gloss of films formed from three test nail topcoat compositions. Specifically a nail topcoat composition (1) comprising an acrylic copolymer having an acid value between 160 mg KOH per gram and about 170 mg KOH per gram but excluding a cellulose ester of at least one C2-C4 organic acid [Example C5]; and a nail topcoat composition (2) comprising an acrylic copolymer having an acid value between 160 mg KOH per gram and about 170 mg KOH per gram and including a cellulose ester of at least one C2-C4 organic acid (CAB) [Example E5, according to embodiments of the invention]. The nail topcoat compositions were otherwise identical (Example E5, which did not have CAB was compensated (“q.s.”) with additional ethyl acetate

Also evaluated was a nail topcoat composition, Example C6, identical to Example similar to Example E5 except that the acrylic copolymer having an acid value between 160 mg KOH per gram and about 170 mg KOH per gram was replaced with an acrylic polymer having an acid value of 0 mg KOH per gram.

Samples were prepared by combining solvents, nitrocellulose, CAB and other plasticizers, other film-forming polymers and other ingredients in a jar. The blend was allowed to let sit 12 to 24 hours. It was then mixed for three minutes with overhead mixer until uniform. The samples were then allowed to sit for 24 hours before testing so any air bubbles would dissipate. The test nail topcoat compositions were evaluated by measuring gloss at 20° and 60°.

To measure gloss, a black and white Laneta card is placed onto a vacuum plate. A film is drawn down using a 6 mil Bird Applicator onto the card and allowed to dry two hours under ambient conditions. A Byk-Gardener Micro-Tri-Glossmeter is powered in its holder and allowed to run autodiagnosis and calibration. If requested, the standard glass plate is cleaned using a glass cleaning cloth and calibration is started by pressing the mode wheel. Readings at 20 and 60 are taken by pressing the start button.

The nail topcoat compositions and gloss results are shown in Table 1.

TABLE 1
Gloss Evaluation

| | EXAMPLE | EXAMPLE | EXAMPLE |
|---|---|---|---|
| COMPONENT | E5 | C5 | C6 |
| Solvents, including acetates | 74% | 74% | 75% |
| Nitrocellulose | 10% | 10% | 10% |
| Acrylic copolymer, acid value | 5% | 5% | — |
| 160-170 (mg KOH per gram) | | | |
| Acrylic copolymer, acid | — | — | 1.5% |
| value 0 (mg KOH per gram) | | | |
| Cellulose acetate butyrate | 1.5% | 0% | 3.5% |
| Other film-forming polymers | 1% | 1% | 1% |
| Other plasticizers | 10% | 10% | 10% |
| Total | 100% | 100% | 100% |
| *apparent errors due to | | | |
| rounding | | | |
| Gloss (20°) | 68.9 | 64.3 | 67.0 |
| Gloss (60°) | 85.6 | 84.3 | 86.0 |

The results indicate surprisingly good gloss performance for the composition with both an acrylic copolymer with an acid value ranging from about 145 and about 170 mg KOH per gram; and a cellulose ester of at least one C2-C4 organic acid.

What is claimed:

1. A composition for use on nails, comprising:
a solvent system comprising at least one volatile, non-aqueous polar solvent;
a water-insoluble nitrocellulose polymer;
an acrylic copolymer with an acid value ranging from about 145 and about 170 mg KOH per gram; and
a cellulose ester of at least one C2-C4 organic acid.

2. The composition of claim 1, wherein the acrylic copolymer has an acid value between about 150 and about 170 mg KOH per gram.

3. The composition of claim 1, wherein the acrylic copolymer has an acid value between about 160 and about 170 mg KOH per gram.

4. The composition of claim 1, wherein the composition is substantially free of colorant.

5. The composition of claim 1, wherein the at least one volatile, non-aqueous polar solvent comprises at least one volatile acetate.

6. The composition of claim 1, wherein the cellulose ester of at least one C2-C4 organic acid is cellulose acetate butyrate.

7. The composition of claim 1, further comprising an ingredient selected from a group consisting of sucrose acetate butyrate and acetyl tributyl citrate.

8. The composition of claim 1, wherein the solvent system comprising at least one volatile, non-aqueous polar solvent comprises ethyl acetate and butyl acetate each in a respective concentration by weight such that a total concentration by weight of ethyl acetate and butyl acetate in the composition is from about 50% to about 90%.

9. The composition of claim 1, wherein the water-insoluble nitrocellulose polymer is nitrocellulose and wherein the nitrocellulose is present in a concentration by weight in the composition that is from about 5% to about 15%.

10. The composition of claim 1, wherein the acrylic copolymer with an acid value ranging from about 145 and about 170 mg KOH per gram is present in a concentration by weight in the composition that is from about 0.5% to about 15% by weight.

11. The composition of claim 1, wherein the cellulose ester of at least one C2-C4 organic acid is cellulose acetate butyrate, and the cellulose acetate butyrate is present in a concentration by weight in the composition that is from about 1% to about 3%.

12. The composition of claim 1, wherein the solvent system comprising at least one volatile, non-aqueous polar solvent comprises ethyl acetate and butyl acetate each in a respective concentration by weight such that a total concentration by weight of ethyl acetate and butyl acetate in the composition is from about 50% to about 90%, wherein the water-insoluble nitrocellulose polymer is nitrocellulose and wherein the nitrocellulose is present in a concentration by weight in the composition that is from about 5% to about 15%, wherein the acrylic copolymer with an acid value ranging from about 145 and about 170 mg KOH per gram has an acid value ranging from greater than 150 to about 170 mg and is present in a concentration by weight in the composition that is from about 0.5% to about 15% by weight, and wherein the cellulose ester of at least one C2-C4 organic acid is cellulose acetate butyrate and wherein the cellulose acetate butyrate is present in a concentration by weight in the composition that is from about 1% to about 3%.

13. A method of treating a nail, comprising:
applying to the nail a composition comprising:
a solvent system comprising at least one volatile, non-aqueous polar solvent;
a water-insoluble nitrocellulose cellulose polymer;
an acrylic copolymer with an acid value ranging from about 145 and about 170 mg KOH per gram; and
a cellulose ester of at least one C2-C4 organic acid.

14. The method of claim 13, wherein the nail has a previously applied color coat formed thereon and the composition is applied atop the previously applied color coat formed on the nail.

15. The method of claim 13, wherein the acrylic copolymer has an acid value between about 160 and about 170 mg KOH per gram.

16. The method of claim 13, wherein the composition is substantially free of colorant.

17. The method of claim 13, wherein the at least one volatile, non-aqueous polar solvent comprises at least one volatile acetate.

18. The method of claim 13, wherein the cellulose ester of at least one C2-C4 organic acid is cellulose acetate butyrate.

19. The method of claim 13, wherein the acrylic copolymer with an acid value ranging from about 145 and about 170 mg KOH per gram is present in a concentration by weight in the composition that is from about 0.5% to about 15% by weight.

20. The method of claim 13, wherein the solvent system comprising at least one non-aqueous polar solvent comprises ethyl acetate and butyl acetate each in a respective concentration by weight such that a total concentration by weight of ethyl acetate and butyl acetate in the composition is from about 50% to about 90%, wherein the water-insoluble nitrocellulose polymer is nitrocellulose and wherein the nitrocellulose is present in a concentration by weight in the composition that is from about 5% to about 15%, wherein the acrylic copolymer with an acid value ranging from about 150 and about 170 mg KOH per gram is present in a concentration by weight in the composition that is from about 0.5% to about 15% by weight, and wherein the cellulose ester of at least one C2-C4 organic acid is cellulose acetate butyrate and the cellulose acetate butyrate is present in a concentration by weight in the composition that is from about 1% to about 3%.
